(19) United States
(12) Patent Application Publication
(10) Pub. No.: US 2005/0158763 A1
(43) Pub. Date: Jul. 21, 2005

(54) Aligned long DNA molecules on templates and methods for preparing

(76) Inventors: Albena Ivanisevic, West Lafayette, IN (US); Dorjderem Nyamjav, Logan, UT (US); Joseph Matthew Kinsella, West Lafayette, IN (US)

Correspondence Address: BARNES & THORNBURG, 11 SOUTH MERIDIAN, INDIANAPOLIS, IN, 46204, US

(21) Appl. No.: 11/015,121

(22) Filed: Dec. 17, 2004

Related U.S. Application Data
(60) Provisional application No. 60/531,352, filed on Dec. 19, 2003.

(51) Int. Cl. C12Q 1/68; C12M 1/34

(52) U.S. Cl. 435/6; 435/287.2; 427/2.11

(57) ABSTRACT

The present disclosure describes methods for aligning nucleic acid molecules in a predetermined configuration on a solid surface. In one illustrative embodiment, DNA is coated with metallic nanoparticles and the coated DNA is positioned on a solid support in a controlled manner.

### CROSS REFERENCE TO RELATED APPLICATIONS

The present application claims priority under 35 U.S.C. § 119(e) to U.S. Provisional Application No. 60/531,352 filed on Dec. 19, 2003, the disclosure of which is incorporated herein by reference.

### US GOVERNMENT RIGHTS

This invention was supported in part by U.S. Department of Energy grant DEFG02-91-ER45 439. The United States Government may have certain rights in the invention.

### TECHNICAL FIELD

This invention pertains to the alignment of DNA on surfaces. In particular, this invention pertains to the alignment of DNA on surfaces by nanolithography.

### BACKGROUND

The ability to control and manipulate molecular scale building blocks into surface structures is crucial in adapting them in micromechanical, microelectronic, bioelectronic and bio-sensing applications. A promising route to achieve such control is through the adaptation of unconventional lithographic approaches. One can further increase the utility of these methodologies by coupling them with templating protocols that are aimed at incorporating inorganic structures among biological moieties.

Scanning probe microscopy (SPM) based techniques have received much attention for use in lithographic methodologies due to the tremendous potential they offer in trying to improve resolution limits. For example, a nanografting approach has been developed where one can displace pre-assembled monolayers of molecules from a surface using an atomic force microscope (AFM) tip (S. Xu, G. Y. Liu, Longmuir 1997 13, 127; S. Xu, S. Miller, P Laibinis, G. Y. Liu, Longmuir 1999, 15, 7244). In another example, dip-pen nanolithography (DPN), where one delivers a collection of molecules to a surface via an AFM tip, was used to pattern many biological and chemical “inks” (R. D. Piner, J. Zhu, F. Xu, S. Hong, C. A. Mirkin, Science 1999, 283, 661; S. Hong, J. Zhu, C. A. Mirkin, Science 1999, 286, 523; S. Hong, J. Zhu, C. A. Mirkin, Longmuir 1999, 15, 7897; C. A. Mirkin, MRS Bull. 2001, 26, 535; and C. A. Mirkin, MRS Bull. 2000, 25, 43).

The challenges one faces in the process of controlling and manipulating molecular scale building blocks include utilization of lithographic strategies that offer flexibility over template designs, ability to transfer template “blue-prints” on electronically important substrates (e.g. GaAs, SiOₓ, and the like), and placement of structures on templates with unique chemical characteristics suitable for (bio)chemical recognition, including highly specific (bio)chemical recognition, subsequent assembly and addressability.

One way to combine the recognition properties of biomolecules with the utility of inorganic materials is to incorporate polyelectrolytes such as nucleic acid molecules into the construction of prototype hybrid structures. In particular, the adjustable size of the DNA molecule, the chemical specificity of its components and its charged nature, make it a promising candidate to serve as a template for the fabrication of nanowires. Examples are reported where DNA has been transformed into a wire via metallization or plating strategies. DNA-templated silver nanowires have been fabricated and control over the electrical properties of the wire were demonstrated (Braun et al. Nature 1998; 391:775-78). More recently, platinium (Adv Mater 2001; 13:1793-97), copper (Nano Lett 2003; 3:359-63), and palladium (Appl Phys Lett 2001; 78:536-38) wires have been made using DNA molecules as a template.

In addition, plasmid DNA was utilized to form ring structures of semiconductor particles such as CdS (Coffer et al. Appl Phys Lett 1996; 25:3851-3). Furthermore, elongated DNA molecules were used to template Au nanoparticles on (i) surfaces coated with designer polymers (Nakao et al. Nano Lett 2003; 3:1391-4), and (ii) silicon surfaces with microfabricated gold electrodes (Harnack et al., Nano Lett 2002, 2, 919-23). Moreover, the utility of using long DNA molecules in the construction of prototype devices has been demonstrated in the past year. Sequence-specific molecular lithography using a long DNA as a substrate (Keren et al. Science 2002, 297, 72-75), and fabrication of a DNA-templated carbon nanotube field-effect transistor (Science 2003; 302:1380-82). The construction of a durable sequence dependent DNA device and a description of its nano-actuating behavior has also been reported (Feng et al. Angew Chem Int Ed 2003, 42, 4342-46).

A common characteristic of conventional strategies is that they rely on random positioning of DNA molecules on a chosen type of surface. Accordingly there is a need for a methodology that allows for more control in the positioning of DNA scaffolds on suitable templates.

### SUMMARY

The present disclosure describes compositions, and methods for preparing such compositions, wherein long DNA molecules are aligned and/or substantially aligned on templates generated by Dip-Pen Nanolithography (DPN). In one illustrative embodiment, DNA molecules are aligned on a template by a combination of lithographic techniques and molecular combing techniques. Conventional molecular combing is a technique that stretches and positions randomly, or with low organization, long DNA molecules onto derivatized surfaces (e.g., silane-treated glass, and like inorganic substrates and surfaces). In another embodiment, the lithographic capabilities of DPN are used to construct or establish a template, and the nucleic acid molecules are stretched and aligned on the template through the use of molecular combing.

In one embodiment of surface templates described herein, surface templates comprising positively and negatively charged regions are fabricated by patterning a polyelectrolyte “ink” onto the surface, such as a SiOₓ surface, and the like. This methodology can be used to localize long DNA molecules in complex architectures such as devices containing regions with different types of materials. DPN allows the positioning of different types of chemical and biological functionalities on various surfaces.

One aspect of the present disclosure is directed to the use of Scanning Probe Microscopy (SPM)-based lithographic techniques to construct high resolution templates with more precisely positioned patterns. DPN is one type of such SPM lithographic approaches, and it has been successfully used to generate surface features composed of thiol molecules, proteins, peptides, viruses, silanes and/or silazanes, inorganic sol-gel precursors, polymers, calixarenes, and DNA on metal and/or semiconductor substrates (Braun et al. Nature 1998, 391, 775-78; and Zhang H et al. Adv Mater 2002, 14, 1472-74). It has been discovered that the versatility of this technique and the promise it has shown in patterning in a site-specific manner in complex architectures (e.g. microfabricated devices) make it suitable for use in fabricating, placing, addressing, and/or miniaturizing templates composed of biological and inorganic components onto a variety of surfaces, including inorganic substrates.

### BRIEF DESCRIPTION OF THE DRAWINGS

FIGS. 1a-1h show DPN patterns of PAH generated onto a SiOₓ surface. FIG. 1a shows the height and FIG. 1b shows the LFM image of a structure patterned by keeping the tip in contact with the surface for 45 sec. These contact-mode images were collected simultaneously with a coated tip, scan size of 20μ×20 μm and a frequency of 3.815 Hz. The height scale in (a) is 20 nm and in (b) is 4 V. FIG. 1c shows the height and FIG. 1d shows the LFM image of lines generated as described in herein and imaged immediately after patterning with a coated tip. These contact mode images were collected simultaneously with a scan size of 15 μm×15 μm and a frequency of 4.07 Hz. The height scale in FIG. 1c is 8 nm and in FIG. 1d is 0.08 V. FIGS. 1e & 1f show representative height and phase images, respectively, collected in TMAFM after the DPN procedure, as described herein. These TMAFM images were acquired simultaneously with a scan size of 6.83 μm×6.83 μm and a frequency of 1.97 Hz. The height scale in FIG. 1e is 8 nm and in FIG. 1f is 10° of phase lag. FIGS. 1g & 1h show representative high-resolution height and phase images, respectively, collected in TMAFM after the DPN procedure, as described in herein. These TMAFM images were acquired simultaneously with a scan size of 2.5 μm×2.5 μm and a frequency of 1.97 Hz. The height scale in FIG. 1g is 5 nm and in FIG. 1h is 5° of phase lag.

FIG. 2 shows the N 1s binding energy region of a high-resolution XPS spectra of a surface coated with a layer of PAH (solid line) and a surface patterned with PAH via DPN (dotted line). The y-axis scale on the left corresponds to the data for the PAH-layer surface and the y-axis scale on the right corresponds to the data for the PAH-patterned surface.

FIGS. 3a-3d show TMAFM images. FIG. 3a shows a TMAFM image of a surface coated with a layer of PAH onto which a drop of DNA solution was adsorbed. The image was collected with a scan size of 1.2 μm×1.2 μm and a frequency of 1.97 Hz. The height scale in FIG. 3a is 10 nm. FIG. 3b shows a TMAFM image of a surface coated with a layer of PAH onto which DNA was combed, as described herein. The image was collected with a scan size of 3 μm×3 μm and a frequency of 1.97 Hz. The height scale in FIG. 3b is 3 nm. FIGS. 3c & 3d show high resolution height and phase images, respectively, of combed DNA on a surface coated with a layer of PAH. The images shown in FIG. 3c and FIG. 3d were acquired simultaneously with a scan size of 1 μm×1 μm and a frequency of 2.54 Hz. The height scale in FIG. 3c is 4 nm and in FIG. 3d is 10° of phase lag.

FIGS. 4a-4e show combing experiments. FIG. 4a shows a schematic of the DPN template used in the combing experiments. FIG. 4b represents the height and FIG. 4c shows the phase images after the combing procedure of a DPN template fabricated as described in the text. Images shown in FIG. 4b and FIG. 4c were acquired simultaneously with a scan size of 8 μm×8 μm and a frequency of 1.97 Hz. The height scale in FIG. 4b is 3 nm and in FIG. 4c is 45° of phase lag. FIGS. 4d & 4e show high resolution height and phase images, respectively, of combed DNA on a DPN template. The images of FIG. 4d and FIG. 4e were acquired simultaneously with a scan size of 4 μm×4 μm and a frequency of 2.18 Hz. The height scale in FIG. 4d is 4 nm and in FIG. 4e is 45° of phase lag.

FIGS. 5a-d show images of DPN patterns and templates. FIG. 5a shows the height and FIG. 5b shows the phase images of a SiOₓ surface containing DPN patterns terminated with a PSS layer. The images were collected simultaneously with a scan size of 5.15 mm×5.15 mm. The height scale in FIG. 5a is 5 nm and in FIG. 5b is 20° of phase lag. FIGS. 5c and 5d are topography images showing parts of the DPN template after the combing of MNP pre-templated DNA on that substrate. The height scale is 12 nm in FIG. 5c and 10 nm in FIG. 5d.

FIGS. 6a & 6b show tapping mode height images of DNA coated with Fe₂O₃ nanoparticles on silicon oxide before enzymatic digestion with BamH1. Both images show typical results when 5 ng of DNA was treated with 5 ng of the nanoparticles for 1 hr prior to molecular combing onto the silicon oxide. The z-scale is 4 nm in both FIGS. 6a & 6b.

FIGS. 7a-7d show tapping mode AFM height images of DNA on the template surface. FIG. 7a shows bare DNA stretched by molecular combing on a modified SiOₓ surface. The line scan shows the height of the DNA to be 0.812 nm. FIG. 7b shows the same strand of DNA as shown in FIG. 7a after it was treated with BamH1. Arrows 1 & 2 indicate areas of the DNA that have been digested. FIG. 7c shows an Fe₂O₃ templated DNA after it was stretched on a clean SiOx surface. The line scan inset shows the height of the templated DNA to be 1.462 nm. FIG. 7d shows the same strand of templated DNA as shown in FIG. 7c after it was treated with 10 units of BamH1. All scale bars indicate 1 micron.

### DETAILED DESCRIPTION

In describing and claiming the embodiments, the following terminology is used in accordance with the definitions set forth below.

As used herein, the terms “nucleic acid” and “polynucleotide” refer to a molecule comprised of a plurality of deoxyribo- and/or ribo-nucleotides or nucleoside subunits. The linkage between the nucleoside subunits can be provided by phosphates, phosphonates, phosphoramidates, phosphorothioates, and like analogs and derivatives thereof, or by known or conventional nonphosphate groups used to link nucleoside subunits, such as peptoid-type linkages utilized in peptide nucleic acids (PNAs), and the like. The nucleic acid can be in either single- or double-stranded form. “DNA” is an abbreviation for deoxyribonucleic acid and refers to a molecule comprised of a plurality of deoxyribonucleotides.

As used herein the term “nanotemplate” refers to a material coated onto a solid support, wherein the smallest dimension, measured parallel to the solid support surface, is within a range from about 10 nm to about 5 um.

As used herein the term “polyelectrolyte” refers to a macromolecule that has a plurality of or multiple charged groups. More specifically a polycation is a macromolecule with a plurality of or many positively charged groups at physiological pH, wherein a polyanion is a macromolecule with a plurality of or many negatively charged groups at physiological pH.

The present disclosure describes nanolithography compositions, nanolithography methods, and compositions prepared using such methods, for controlled positioning of a DNA scaffold on a suitable template. More particularly, the present disclosure describes methods for aligning long chain polyelectrolyte molecules in a desired configuration on a solid surface. The methods comprise the steps of first localizing the polyelectrolyte molecules on a nanotemplate formed on a solid surface, and then controlling the direction that the polyelectrolyte molecules are elongated to provide a scaffold structure comprising the polyelectrolyte in a desired configuration. Additional components can then be bound or templated onto the polyelectrode scaffold to provide the scaffold with desired functionalities. In one embodiment, the polyelectrolyte scaffold can be complexed with nanoparticles, wherein the nanoparticles comprise metal elements, to create nanowires.

In accordance with one embodiment, the polyelectrolyte molecule that is aligned on a solid surface is a nucleic acid. In accordance with one embodiment, a method of aligning nucleic acid molecules in a desired configuration on a solid surface is provided. The method comprises the steps of localizing the nucleic acid molecules on a nanotemplate that is formed on a solid surface. After the DNA has been placed in contact with the nanotemplate, the nucleic acid molecules are elongated in a controlled direction to provide elongated nucleic acid molecules in a desired configuration. In one embodiment the aligned nucleic acid sequence is DNA.

The nucleic acid sequence can be localized onto a nanotemplate through the use of electrostatic interactions. Accordingly, in one embodiment the nanotemplate comprises a polycationic surface to localize the nucleic acid on the nanotemplate. In one embodiment the polycation used to form the nanotemplate is poly(allylamine hydrochloride) (PAH), however, it is appreciated that many different polycations can be used herein. The nanotemplate is coated onto standard or conventional semiconductor materials. In one embodiment a solid support is provided comprising a group III-V or group II-VI semiconductor material that is terminated with an oxide layer. In one embodiment the solid support comprises silicon oxide.

The nanotemplate can be coated onto the semiconductor solid support using standard lithographic techniques including the use of dip-pen nanolithography. The templates can be formed in a variety of different shapes, and it is appreciated that the selection of the shape may be based at least in part on the desired application of the final product. In one embodiment the template shape is selected from patterns with three different shapes (dots, squares, and lines) that range in size from about 50 nm to several micrometers (FIGS. 1a-1h). The fabrication of each of these types is briefly described as follows, using PAH as an illustrative embodiment of a polycationic template. Dots were created by keeping the poly(allylamine hydrochloride) (PAH) coated contact mode tip in contact with the surface for a given period of time. The dot shown in FIGS. 1a and 1b is made by keeping the tip in contact with the surface for 45 sec and imaged in contact mode with the same tip. This preparation and preliminary imaging was performed to verify the imaging prior to duplicating a given feature a number of times across the surface, illustratively to form a template. To minimize further deposition of “ink” this verification was done at a high scan rate (tip speed 152.6 μm/s) resulting in the appearance of artificial streaks on the sides of the image. Square patterns were made by scanning with the PAH coated tip in a 1:1 aspect ratio. The scan speeds used were between 3.97 and 19.7 μm/s. Upon the completion of a scan cycle, the tip was moved by changing the X and/or Y offset values manually. All of the vertical polymer lines, see FIGS. 1c-1h, were created by scanning with the coated tip with a scan angle of 0°, scan size of 5 μm, an aspect ratio of 1:256, and a scanning rate of 1.97 Hz (scanning speed 19.7 μm/s). The lines were drawn manually by changing the Y offset values using increments of 1-1.5 μm.

The resulting patterns on the surface were then examined by lateral force microscopy (LFM) and tapping mode AFM (TMAFM) with a scanning angle of 90°, a scanning rate of 4 or 5 Hz, and scan sizes of either 15 μm or 20 μm. The patterns exhibited consistent dimensions and chemical composition based on the LFM or phase contrast. From all the experiments performed, it is appreciated that immediately after patterning, one facile way to verify the patterning process is to image with either a clean or coated tip in contact mode. Upon washing the patterned surface with deionized (DI) water and imaging it in TMAFM, it is may be difficult to identify nanometer-sized features from height images (FIGS. 1e and 1g). However, phase images generally show the pre-programmed structures and can be used to estimate sizes of the features. Residual salt from washing the surfaces can cause “grainy” images. The polymer lines shown in FIGS. 1f and 1h had widths of 150 nm and 209 nm respectively. Illustratively, imaging the generated templates is in TMAFM; it is appreciated that the success of stretching long and soft DNA molecules on the surface (as described hereinbelow) may be advantageously evaluated in a non-destructive fashion.

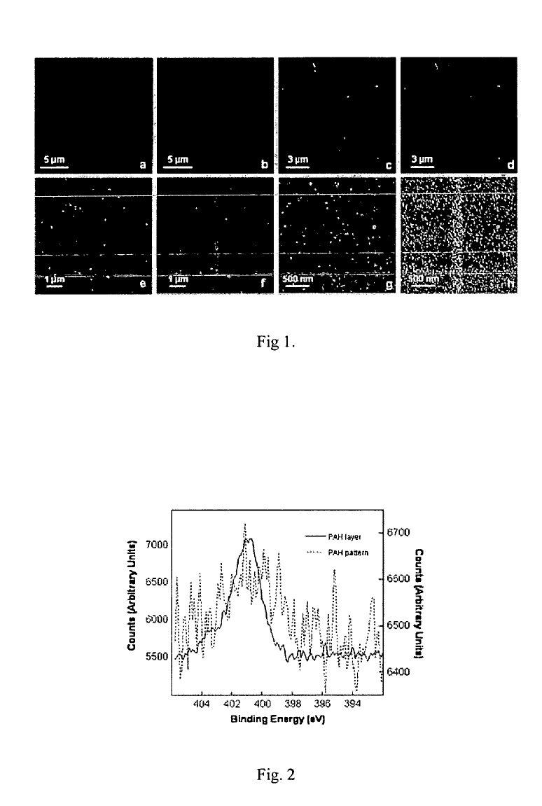

In addition to the AFM characterization of the patterning process, the delivery of the polyelectrolyte to the surface via DPN was verified by performing scanning X-ray photoelectron spectroscopy (XPS) experiments. For this purpose, a SiOₓ surface with a micrometer size alignment feature was used and large (15 μm×15 μm) multiple, PAH patterns, were generated next to one another to cover an area of 105 μm×105 μm next to the alignment marker. Spectra from this restricted region were collected. Survey and high-resolution scans were obtained from clean SiOₓ surfaces, SiOₓ surface coated with a thin film of PAH (see Example 1), and SiOₓ surfaces patterned with PAH via DPN. All surfaces were rinsed with DI water and dried under nitrogen prior to the XPS experiments. The N 1s peak was used as a way to identify the presence of PAH on the surface. High-resolution scans showed no evidence of nitrogen on a clean SIOx substrate. The high-resolution N 1s scans for the other two surfaces are shown in FIG. 2. A characteristic strong peak at 400.88 eV was observed on the surface coated with a layer of PAH. A diminished response at the same binding energy was detected on the surface patterned with PAH via DPN. Because of the small amount of PAH deposited onto the surface during the DPN procedure, the signal-to-noise ratio decreased significantly in this XPS spectrum. Other high-resolution scans on these two surfaces confirmed the presence of carbon, oxygen, silicon, and chlorine.

Prior to stretching long DNA molecules on the DPN templates the combing procedure was performed on surfaces modified with a layer of PAH. In one embodiment lambda DNA was bound to the polycationic template. However any DNA sequence can be used, and more particularly, in one embodiment recombinant DNA sequences are used that comprise one or more unique endonuclease restriction recognition sites. Such endonuclease restriction sites allow for the DNA strands to be cleaved after the sequences have been bound to the template, and thus provide an added capability to design DNA scaffold patterns on the template surface.

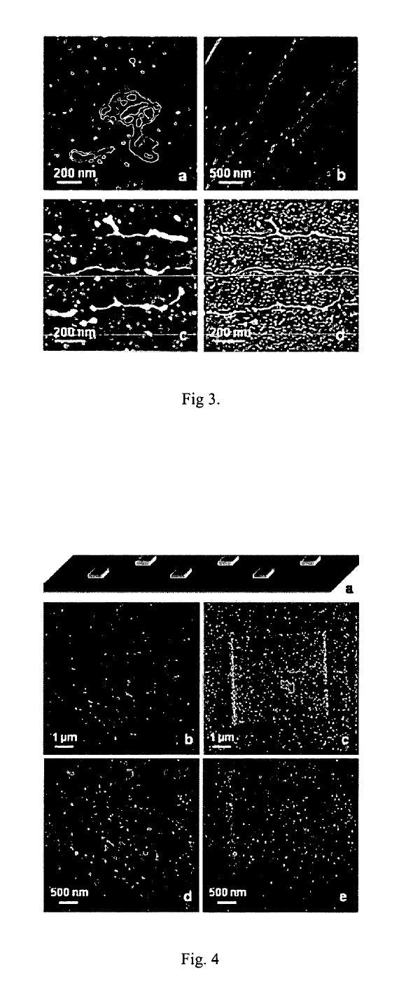

As described in Example 1 a DNA solution was prepared by dissolving 6 ng of λ-DNA, non-methylated from E. coli host strain GM 119, Sigma-Aldrich in 1 mL of 10 mM Tris(hydroxymethyl)aminomethane (Tris), 1 mM ethylenediaminetetraacetic acid (EDTA), pH 8.0 (TE buffer, Aldrich), or in 1 mL of 1 mM Tris, 0.1 mM EDTA, pH 8.0 and aligned on the template. In solution, λ-DNA molecules are coiled to maximize their entropy and keep their coiled shape once deposited onto substrates, see FIG. 3a. The height of the coiled molecules was 2.45±0.36 nm, which was calculated from five randomly positioned line profiles. However, upon utilizing a modified molecular combing procedure (see Example 1), DNA strands can be extended along a positively charged surface modified with a layer of PAH (FIGS. 3b-3e).

Molecular combing of the DNA was performed on the patterned templates after each surface was rinsed, dried, and examined to verify that pre-programmed patterns were generated. In a typical experiment, the silicon piece was placed on a XYZ translation stage, which is part of a contact-angle apparatus (see Example 1). The needle of the syringe was filled with DNA solution, and brought within a vertical distance of 1-2 mm. A droplet of DNA solution was suspended between the tip of the needle and the substrate surface using the droplet regulator on the syringe equipped with a micrometer. Subsequently, the substrate was moved in the X or Y direction using the translation stage and keeping the droplet of DNA solution suspended between the syringe needle and the substrate. The translation stage was moved at a speed of 30-50 μm/s. The long DNA strands extend parallel to the direction of the liquid drop movement. In cases when the DNA solution was prepared in 10 mM Tris buffer, the surfaces were rinsed with DI water and dried with nitrogen after the combing procedure. When solutions of DNA were prepared in 1 mM Tris buffer at the end of the combing procedure, no additional treatment was done, other than drying it with nitrogen. In all cases, the templates were examined by TMAFM within minutes of completing the above protocol.

Accordingly, by controlling the direction in which the liquid drop is moved, one can control the direction that the DNA is stretched/extended. Furthermore, through the use of a DPN deposited nanotemplate, that comprises multiple spots of a polycation, DNA strands can be extended in a first direction (from a first polycation spot to a second polycation spot) and then extended in a second direction (from the second polycation spot to a third polycation spot) by controlling the movement of the liquid drop comprising the DNA sequences. In this manner nucleic acid strands can be positioned on the nanotemplate in multiple directions and in a controlled fashion to prepare various scaffold patterns.

The measured height of the DNA molecules was 0.58±0.02 nm, calculated from eight randomly positioned line profiles. Furthermore, the stretched strands can be visualized from height as well as phase images, FIGS. 3c and 3d, respectively. From these high resolution scans the height of the DNA molecules was 0.73±0.01 nm and was calculated from four randomly positioned line profiles. The height values for the stretched DNA molecules are higher than expected. This may be due to the formation of secondary structure and are most likely convoluted due to the surface roughness of the PAH coated substrate used (root mean square, RMS=0.18±0.03 nm, extracted from a 1 μm×1 μm area of the images).

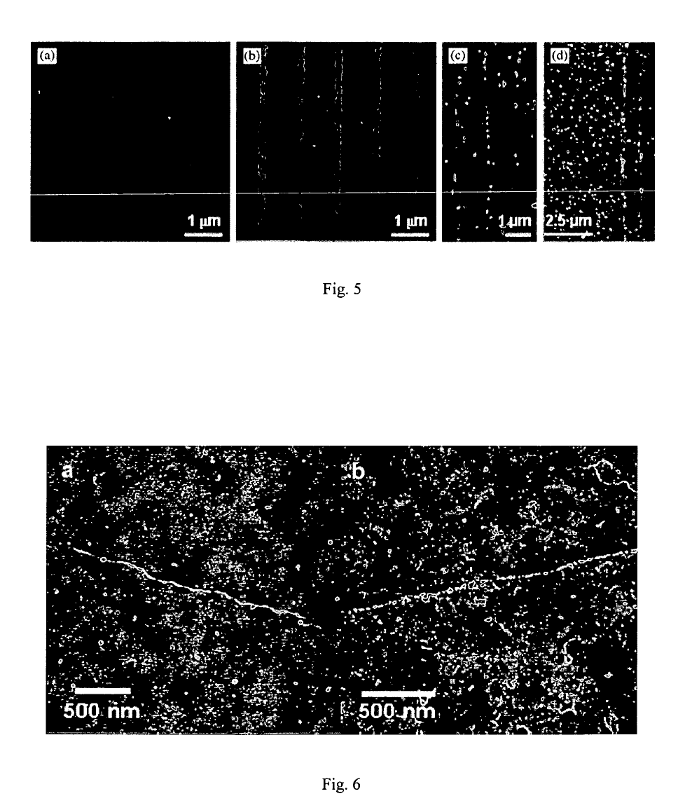

TMAFM images show that the DNA molecules were elongated selectively on the surface (see FIGS. 4 and 5). In one type of a template design (FIG. 4a), 4 μm×4 μm PAH patterns were multiplied across the surface. When such large patterns are utilized in the combing procedure, DNA molecules can bridge and stretch across patterns. FIG. 4c shows the entire PAH pattern in phase and numerous DNA molecules stretched across the square in the height image (FIG. 4b). When one zooms in on the DNA molecules inside the pattern (FIGS. 4d and 4e) it is observed that the long DNA strands elongate across the entire pattern and towards others positioned around the one imaged. Without being bound by theory, it is believed that when the combing procedure is performed, one end of the negatively charged DNA molecule binds to the positively charged PAH pattern and the surface tension force exerted on the DNA molecule at the moving interface of the air-water media is sufficient enough to stretch it but weak enough not to break the bond.

The experiments described in Example 1 indicate that the DPN template directs the placement of the DNA molecules on a desired surface location. However, the direction along which such molecules elongate is determined by the direction of the droplet movement. This is demonstrated by utilizing templates with nanometer size features, wherein reproducible results show substantially complete stretching of more than about 65% of the DNA strands. It is appreciated that the present methodology may be advantageous because it provides the ability to perform additional lithographic steps upon combing of the DNA molecules without destroying them, allows for the positioning of other inorganic and organic structures on the same template, and allows lithography to be performed on different types of materials with the same tool. Furthermore, polyelectrolyte inks can be patterned by DPN on hard and soft surfaces and used to guide the assembly of nanoparticles modified by the layer-by-layer approach.

In accordance with one embodiment nucleic acid sequences localized on a nanotemplate provide a scaffold whereby additional components can be added to form nanowires or other components for the assembly of microelectronic circuits. In one embodiment the scaffold bearing device comprises a semiconductor solid support coated with a surface nanotemplate to provide well-defined positively and negatively charged regions, wherein a nucleic acid sequence is localized and stretched across the surface of the nanotemplate. In one embodiment the nucleic acid sequence is complexed with a plurality of nanoparticles to metalicize the nucleic acid sequence. Typically the nanoparticles are bound to nucleic acids via electrostatic interactions, however other binding interaction, such as hydrophobic/hydrophilic, ionic, hydrogen bonding and the like can also be used to associate the nanoparticle with the nucleic acid. Accordingly, in one embodiment a dip-pen lithography technique is used to deposit a nanotemplate on a solid surface and metalicized DNA sequences are bound to the nanotemplate in a desired pattern. The nanotemplate may be applied as a continuous coated pattern on the solid surface or the nantemplate may be applied as a series of distinct patches, spots, lines or other series of distinct shapes.

In accordance with one embodiment nucleic acid sequences are bound to nanoparticles prior to being localized onto the nanotemplate. Furthermore, in this embodiment the polycationic template is typically coated with a polyanionic compound prior to localizing the nanoparticle coated DNA molecules on the nanotemplate. In one embodiment the nanoparticles comprise one or more metallic elements that are bound to the nucleic acid sequence through electrostatic interactions. In one embodiment the nanoparticles comprise electrically conductive material and included elements selected from the group consisting of Au, Ag, Cu, and Pt. In another embodiment the nanoparticles comprise magnetic material, including for example Fe₃O₄.

In another embodiment, nanoparticle coated DNA sequences are localized onto a template surface and are cleavable or cleaved by restriction endonucleases. Accordingly these endonucleases can be used to cut the nanowires (the metallic nanoparticle coated DNA) described herein. In accordance with one embodiment a nanoparticle coated DNA sequence is provided wherein the DNA sequence has one or more endonuclease restriction recognition sites located at a predetermined site on the DNA sequence. Advantageously, the inclusion of multiple unique restriction sites on the DNA will allow the clipping of different lengths of the DNA nanowire as needed, or alternatively, the simultaneous use of two unique restriction enzymes will allow for the removal of one or more segments of the DNA nanowire. It is appreciated that the use of restriction enzymes may add another layer of flexibility in designing and building nanocircuitry.

In accordance with one embodiment a method of localizing a nanoparticle coated nucleic acid molecules in a desired configuration on a solid surface is provided. The method comprises the steps of preparing a nanotemplate by DPN coating a polycationic polymer on a solid surface, coating the nanotemplate with a polyanionic polymer, localizing the nucleic acid molecules on the nanotemplate formed on the solid surface, and elongating the nucleic acid molecules in a controlled direction to provide elongated nucleic acid molecules in a desired configuration. In a further embodiment the method includes the step of cleaving the nanoparticle coated nucleic acid molecules with an endonuclease that cleaves the nucleic acid sequence at a predetermined location.

One aspect of the present disclosure is directed to compositions comprising molecular scaffolds prepared in accordance with the described method. In one embodiment a composition is provided comprising nucleic acid molecules arranged on a nanotemplate, wherein the naontemplate comprises a polyelectrolyte polymer formed on a solid support surface. More particularly, the nucleic acid molecules have been elongated and positioned on the nanotemplate in a desired arrangement using combing techniques. In one embodiment the nucleic acid molecules are modified by binding nanoparticles to the nucleic acid. In one embodiment the nucleic acid is a DNA molecule and the DNA is coated with metallic nanoparticles. The DNA can be bound to nanoparticles either prior to or after the DNA is localized on the nanotemplate.

In one embodiment the nanotemplate is applied to a semiconductor solid support, wherein the semiconductor solid support is a hard or soft surface. Suitable semiconductor surfaces include but are not limited to conventional semiconductor surfaces, group III-V and group II-VI semi-conductor type material terminated with an oxide layer, and the like. In one embodiment the solid support comprises SiO_{X}.

In accordance with one embodiment the molecular scaffolds described herein comprise a solid support, (wherein the solid support comprises a semiconductor type material) a nanotemplate formed on and adhered to the solid surface, (wherein the nanotemplate comprises a polycationic electrolyte polymer), and a nanoparticle coated nucleic acid sequence located, stretched, and adhered to the nanotemplate in a desired orientation. In one embodiment the nanotemplate comprises a polycation, and a layer of an polyanion coating the exposed polycation surface of the nanotemplate. In one embodiment the nucleic acid comprising the molecular scaffold is a DNA sequence. In one embodiment the DNA sequence is a recombinant DNA sequence that has been modified to include unique restriction endonuclease recognition sites at predetermined positions of the DNA sequence. In one embodiment the restriction endonuclease and its corresponding recognition site used in the recombinant DNAs are selected from the type-II endonucleases, and include but are not limited to EcoRI, BamHI, HindIII, XbaI, XhoI, SalI, PstI, and Kpn. The inclusion of a unique restriction site in the DNA sequence allows the DNA, even when coated with nanoparticles, to be cleaved after the DNA has been localized and positioned on the template. Such cleavage of DNA nanowires or other molecular scaffolds provides an additional level of manipulation for constructing desired structure.

In accordance with one embodiment a kit is provided for preparing the molecular scaffolds described herein. In one embodiment the kit comprises a DNA molecule that is coated with nanoparticles, wherein the nucleic acid further comprises a unique restriction endonuclease recognition site. In one embodiment the nanoparticle coated DNA sequence comprises a plurality of unique restriction endonuclease recognition sites positioned at predetermined distances from one another. Accordingly, the kit allows for the creation of an appropriately sized nanoparticle coated DNA sequence from the kit's stock solution of unique restriction endonuclease bearing nanoparticle coated DNAs by contact of the DNA with a selected endonuclease. Cleavage of the DNA sequence can take place either before or after the nanoparticle coated DNA is localized on a nanotemplate. In one embodiment the kit further comprises a solid support and a nanotemplate formed on and adhered to the solid support. In one embodiment the solid support comprises a semiconductor type material (such as SiO_{X}) and the template comprises a series of polycationic polymer spots or polyhedral shapes distributed across the surface of the solid support. In one embodiment the template comprises an evenly distributed pattern of polycationic polymer deposits. In one embodiment the external surface of the polycationic polymer deposits is coated with a polyanionic polymer.

In one embodiment a kit for aligning nucleic acid sequences on a solid surface is provided, wherein the kit comprises a nucleic acid sequence, a solution comprising nanoparticle and a solution of a polycation, wherein the polycation is suitable for use in dip-pen nanolithography. In one embodiment the nanoparticles comprise a metal, and in one embodiment the metal is selected form the group consisting of Fe, Au, Ag, Pt, and Cu. In one embodiment the kit comprises a nucleic acid wherein a plurality of nanoparticles are bound to the nucleic acid sequence. In one embodiment the nucleic acid is DNA and in a further embodiment the DNA includes one or more unique endonuclease restriction site located at a predetermined distance from a first end of the nucleic acid sequence. In another embodiment the kit further comprises a solution of a polyanion.

In one embodiment a kit is provided comprising a recombinant nucleic acid sequence in linear form and including one or more unique restriction endonuclease recognition sites, and a solid support, wherein the solid support has a nanotemplate formed on and adhered to the solid support. In one embodiment the solid support comprises a semiconductor type material (such as SiO_{X}) and the template comprises a series of polycationic polymer spots or polyhedral shapes distributed across the surface of the solid support. In one embodiment the nucleic acid sequence is DNA and the template comprises an evenly distributed pattern of polycationic polymer deposits. In one embodiment the DNA is coated with metallic nanoparticles and the external surfaces of the polycationic polymer deposits are coated with a polyanionic polymer.

### EXAMPLES

### Example 1

Aligning DNA on a PAH Template

A SiOₓ surface with a micrometer size alignment feature was used and large (15 μm×15 μm) multiple, PAH patterns, were generated next to one another to cover an area of 105 μm×105 μm next to the alignment marker. The templates were prepared with three different shapes: dots, squares and lines, and range in size from 50 nm to several micrometers.

Prior to the patterning procedure, surfaces were prepared in the following fashion: 1 cm×1 cm silicon pieces were cut from a 4 inch SiOx wafer (Wafer Net, CA) using a diamond scribe. Every piece was cleaned with piranha solution (H₂SO₄/H₂O₂ at 3:1, vol./vol.) for 20 min, rinsed with DI water and dried with a stream of nitrogen. This treatment results in a negatively charged “hard” surface used for patterning (S. Hong, J. Zhu, C. A. Mirkin, Longmuir 1999, 15, 7897). In addition, cleaned silicon pieces were coated with a layer of poly(allylamine hydrochloride) (PAH) Mw=50,000 (Aldrich) using a conventional procedure described by Chen and McCarthy [40], and by Chen et al., W. Chen, T. McCarthy, Macromolecules 1997, 30, 78. Briefly, a clean silicon substrate was immersed into a 10 mg/mL PAH solution (0.5 M NaCl water solution was the solvent), for 20 min at room temperature followed by rinsing with DI water 3 times and drying with nitrogen. Contact-angle measurement was performed using a Tantec, Inc. contact-angle meter (Model CAM-PLUS MICRO) and utilizing the half-angle method. The static contact angles were measured within 30 s of depositing a drop of water. The contact angles were 20° for clean SiOx and 22° for PAH-terminated surfaces, and are consistent with similar experiments in the literature. Furthermore, the deposition of a PAH layer was verified via this method as well as DPN through XPS experiments.

All DPN patterning was performed under ambient conditions, where the temperature range was 20-26° C. and the humidity was between 21-40%, using Multi-Mode SPM from Digital Instruments, equipped with a Nanoscope software system. The instrument was placed in a home-built chamber that allowed for the control of environmental conditions during the fabrication process. The “V”-shaped triangular contact (Model MSCT-AUHW) and single-beam-shaped tapping mode tips (Model OMCL-AC160TS-W2) were purchased from Veeco Instruments and had spring constants of 0.05 N/m and 42 N/m, respectively. Contact-mode tips that were used for lithography experiments were coated with PAH, by dipping them into solutions of PAH for 2-5 s. The ink solution was prepared by dissolving 2 mg of PAH in 1 mL solution of 0.5 M NaCl in water. After being blown dry with compressed nitrogen, the tips were immediately mounted into a tip holder and used in the DPN procedure. Contact mode was used to do the patterning and the imaging was done with either contact or tapping mode AFM. All LFM images reported here were collected at a scan angle of 90°. The LFM image represents the change in twisting of the cantilever beam, mainly caused by frictional force between the tip and the surface. The contrast of the LFM image depends on the scan angle and it is reversed when scan angle is changed to 270°.

For all combing experiments a DNA solution prepared by dissolving 6 ng of λ-DNA, non-methylated from E. Coli host strain GM 119, Sigma-Aldrich in 1 mL of 10 mM Tris(hydroxymethyl)aminomethane (Tris), 1 mM ethylenediaminetetraacetic acid (EDTA), pH 8.0 (TE buffer, Aldrich) or in 1 mL of 1 mM Tris, 0.1 mM EDTA, pH 8.0 was used. In solution, λ-DNA molecules are coiled to maximize their entropy and keep their coiled shape once deposited onto substrates. Molecular combing (Bensimon et al., Science 1994, 265, 2096; and Michalet et al., Science 1997, 277, 1518) of the DNA was performed on the patterned templates after each surface was rinsed, dried, and examined to verify that pre-programmed patterns were generated.

The silicon piece was placed on a XYZ translation stage, which is part of the contact-angle apparatus. The needle of the syringe was filled with DNA solution, and brought within a vertical distance of 1-2 mm. A droplet of DNA solution was suspended between the tip of the needle and the substrate surface using the droplet regulator on the syringe equipped with a micrometer. Subsequently, the substrate was moved in the X or Y direction using the translation stage and keeping the droplet of DNA solution suspended between the syringe needle and the substrate. The translation stage was moved at a speed of 30-50 μm/s. DNA strands were extended along the positively charged PAH template (FIGS. 3b-3e). The long DNA strands extend parallel to the direction of the liquid drop movement.

In cases when the DNA solution was prepared in 10 mM Tris buffer, the surfaces were rinsed with DI water and dried with nitrogen after the combing procedure. When solutions of DNA were prepared in 1 mM Tris buffer at the end of the combing procedure, no additional treatment was done, other than drying it with nitrogen. In all cases, the templates were examined by TMAFM within minutes of completing the above protocol.

TMAFM images show that the DNA molecules were elongated selectively on the surface (FIGS. 4 and 5). In one type of a template design (FIG. 4a), 4 μm×4 μm PAH patterns were multiplied across the surface. When such large patterns are utilized in the combing procedure, DNA molecules can bridge and stretch across patterns. FIG. 4c shows the entire PAH pattern in phase and numerous DNA molecules stretched across the square in the height image (FIG. 4b). When one zooms in on the DNA molecules inside the pattern (FIGS. 4d and 4e) it is observed that the long DNA strands elongate across the entire pattern and towards others positioned around the one imaged.

These results show that the DPN template directs the placement of the DNA molecules on a desired surface location. However, the direction along which such molecules elongate is determined by the direction of the droplet movement. We demonstrate this by utilizing templates with nanometer size features. The experiment is reproducible and results in substantially complete stretching of more than about 65% of the DNA strands.

### Example 2

Aligning Nano-Templated DNA on a DPN Template

Materials.

Poly(allylamine hydrochloride) (PAH), with molecular weight of MW of 70,000, poly(sodium-4-styrenesulfonate) (PSS) of MW 70,000; 8-phage single stranded DNA, non-methylated from Escherichia coli host strain GM 119, FeCl₂.4H₂O and FeCl₃.6H₂O, were purchased from Aldrich. Polished 4 inch SiOₓ wafers with 500 nm thermally evaporated oxide layer were obtained from Wafer Net Inc, CA and cut into smaller pieces. Solutions of 1 M Tris, 0.1 M EDTA, pH=8.0 buffer and tetramethylammonium hydroxide (TMAH), (25% of weight in water) were used as received from Aldrich.

Surface Preparation.

Pieces of silicon (1 cm×1 cm) were cleaned with a piranha (H₂SO₄/H₂O₂ at 3:1, vol./vol.) solution for 20 min, rinsed thoroughly with dionized (DI) water, sonicated in ethanol, and dried under a stream of nitrogen. The freshly cleaned negatively charged silicon surfaces were used for the DPN patterning. In some experiments, the layer-by-layer (LbL) method (Decher et al., Macromolecules 1993; 26: 7058-63) was implemented to deposit multilayer polyelectrolyte films by alternatively adsorbing PAH (polycations) and PSS (polyanions) on the freshly cleaned silicon surface. Solutions of PAH and PSS were prepared with concentrations of 10 mg/mL in a water solution of 0.5M NaCl. To carry out the LbL assembly, the substrates were immersed into a solution of polycations or polyanions for 20 min, followed by rinsing with water and drying with nitrogen.

DPN Patterning.

Multi-Mode™ SPM from Digital Instruments, equipped with a Nanoscope software system, was used for all of the DPN experiments and atomic force microscopy (AFM) imaging. The SPM probes used in the experiments, the “V” shaped triangular contact (Model # MSCT-AUHW) and single beam shaped tapping mode tips (Model # OMCL-AC16OTS-W2) were purchased from Veeco Instruments, CA and had spring constants of 0.05 N/m and 42 N/m, respectively.

Contact Mode AFM was Used For the DPN Patterning.

All the experiments were performed under ambient conditions where the temperature and humidity ranged from 20 to 24° C., and from 25% to 35%, respectively. Throughout the patterning experiments the SPM instrument was placed in a home-built glove box equipped with temperature and humidity controls. The ink solution in this study was 0.3 mM of PAH in a water solution of 5 mM of NaCl. Ink solutions of PAH with concentrations of 0.1-10 mM can also be successfully used in DPN experiments. The tips were coated with PAH by briefly (5-60 s) placing them into an ink solution, followed by drying with nitrogen. The tips were immediately mounted onto the AFM tip holder and utilized in the lithography experiments. The patterns consisting of 20-40 lines with lengths of 5 or 10 mm were generated using a scan aspect ratio of 256:1. During patterning, the scan parameters were fast scan lines of 512 with scan rates of 1.97 Hz (tip speed of either 19.7 mm/s when a 5 mm scan sizes were used or 39.4 mm/s for 10 mm scan sizes). The tip was moved by manually changing the Y offset value by increments of 1-2 mm, while the scan direction was kept at 01. Either lateral force microscopy or tapping mode AFM (TMAFM) with clean tips was used to verify the DPN patterns, prior to using the substrates for further experiments. Depending upon the experimental conditions, the imaging was carried out with scan speeds of 1-4 Hz. For certain experiments, the patterned substrates were exposed to a 10 mg/mL solution of PSS (the solvent was 0.5M NaCl water solution). After 20 min of incubation, the substrates were rinsed with water and dried with nitrogen. As a result, the DPN generated patterns on the surface were coated with an additional polyelectrolyte layer of PSS which makes the lithographically generated features negatively charged.

Magnetic Nanoparticles (MNP).

Fe₃O₄ MNP were synthesized based on a procedure by Berger et al. (J Chem Edu 1999; 76:943-8). Briefly, 4 mL of 2M FeCl₃ and 1 mL of 2M FeCl₂ in 2M HCl were mixed and stirred for 20-30 min. Drops of 50 mL of 2M NH₃ were added to the mixture, which was continuously stirred. As a result, the mixture turns dark brown and a black magnetite precipitate forms at the bottom of the flask. A magnet was placed under the flask allowing the magnetite to settle for 5-10 min. The magnetite was separated from the excess ammonia by washing and re-dispersing it in DI water. This cycle was repeated 3-4 times. Subsequently, the MNP were suspended in a 4 mL solution of TMAH. This particle solution was diluted 10-100 times in TMAH, centrifuged and filtered through a 0.2 mm pore-sized filter. The size distribution of the filtered MNP was determined from TMAFM images. The sample for TMAFM evaluation was prepared by placing a droplet of MNP solution on a PSS-terminated surface for 5 min. The samples were rinsed with DI water and blown dry with a stream of nitrogen.

Pre-Templated DNA with MNP.

The DNA molecules were templated by incubating them in a solution of MNP. First, a DNA solution was prepared by dissolving 25 ng of as purchased DNA into a 1 mL in 1 mM Tris, 0.1 mM EDTA, pH=8.0 buffer. Second, the filtered or unfiltered MNP stabilized by TMAH were diluted several times. Last, a mixture was prepared by mixing the DNA and MNP solutions described above in a 1:5 (v:v) ratio. This mixture was “aged” for 30 min on the laboratory bench at room temperature prior to use.

Molecular Combing.

Molecular combing of DNA or pre-templated DNA with MNP on DPN templates was performed in accordance with Nyamjav et al., Adv Mater 2003; 15:1805-9. Briefly, a contact angle meter from Tantec, Inc (Model CAMPLUS MICRO) was used where the substrate containing the DPN patterns was placed on a XYZ translation stage. The needle of the syringe, fixed above the translation stage, was filled with DNA or pre-templated DNA with MNP solution, and brought within a vertical distance of 1-2 mm from the substrate surface. A droplet of the solution was suspended between the tip of the syringe needle and the substrate surface using a regulator on the syringe equipped with a micrometer. The substrate was moved in the X or Y direction using the translation stage while keeping the droplet of the solution suspended between the needle and the substrate. The translation stage was moved at a speed of 30-100 mm/s. As the droplet moves across the surface the moving air-water interface straightens the DNA whose one end is bound to the surface via strong electrostatic interactions. In cases when the pretemplated DNA with MNP solution was used, the droplet was allowed to stand in a patterned area, terminated with a PSS layer, for 3 min before it was moved across the surface. All samples were rinsed thoroughly with DI water, dried under a stream of nitrogen, and examined by TMAFM within minutes of finishing the combing procedure.

Results.

The importance of one-dimensional arrays of ordered nanoparticles (or nanowires) has been recognized in many applications such as optoelectronic or magnetic storage platforms (see Wurtz et al., J Phys Chem B 2003; 107:14191-8; McConnell et al., J Phys Chem B 2000; 104:8925-30; and Dickson R M, Lyon L A. J Phys Chem B 2000; 104:6095-8). In particular, arrays of MNP with appropriate height, spacing and shape can be potentially incorporated in a variety of biosensing and bioelectronic devices (Lee et al., J Phys Chem B 2002; 106:2123-6). Furthermore, when coupled with the proximity of an appropriate metal surface, they can exhibit an enhancement in their magnetic circular dichroism. When Fe₃O₄ MNP are cast on a surface coated with PSS, a wide range of particle sizes can be seen. The sizes of the particles were extracted from TMAFM images and represent an enlargement due to the interaction of the AFM tip with the sample.

For the present studies it was advantageous to size the particles by AFM since it was desirable to make comparisons between bare DNA and DNA templated with MNP. Upon centrifugation and filtration one can select particles with sizes ranging from 1 to 5 nm. Dilute solutions of the particles were dried on surfaces coated with PSS prior to the collection of TMAFM images in order to avoid aggregation on the surface. A total of 200 particles were measured in 10 different TMAFM images of 1×1 mm² and the mean diameter of the MNP was determined to be 3.171.1 nm. In general, a fresh batch of MNP was synthesized prior to each experiment and the same approximate height was observed each time. For all of the templating experiments the dilutions of MNP needed in order to obtain optimal templating of the DNA molecules was determined empirically by either of two methods described herein.

Two-Step Templating of Stretched DNA.

The recognition properties of the Fe₃O₄ MNP were first tested prior to using any templates generated via DPN. Initially, a droplet of DNA solution was combed randomly on a surface terminated with a PAH layer. The height of the DNA stretched on the surface by this method was measured to be 0.58±0.02 nm. Upon verifying that the DNA molecules were stretched, numerous concentrations of MNP were tested by applying a drop of diluted MNP solution on top of the surface. Various incubation times were also investigated; allowing the drop to stay on the surface for periods longer than 5 min resulted in no appreciable differences in the templating and only contributed to non-specific binding to the surface terminated with PAH. The combed DNA strands on the surface were templated with MNP most optimally when a solution of as synthesized, centrifuged and filtered MNP was diluted with TMAH in a ratio of MNP:TMAH=1:10 (v:v). A drop of MNP was left on the substrate with the combed DNA for 5 min. The sample was then rinsed and dried with a stream of nitrogen and characterized with TMAFM. The imaged MNP teplated DNAs revealed a variable amount of templating on each one. Furthermore, the topography images showed that within a given DNA strand the templating with the MNP can also be partial thus leaving parts of the DNA free of any MNPs. The heights of the coated and uncoated parts of the DNA are 5.8 and 0.88 nm, respectively, extracted from line profiles drawn randomly across these regions of the same DNA strands. The difference of 4.9 nm agrees with the expected size range of the MNP. Interparticle distances were determined to be about 10-15 nm. Structures with similar interparticle distances have been used in experiments that were aimed at determining the origins of enhanced magnetic circular dichroism (Shemer et al., J Phys Chem B 2002; 106:9195-7). In comparison with other approaches that rely on stretching DNA and subsequently templating with metals, the wires prepared herein display similar densities and heights of nanoparticles (Richter et al., Appl Phys A 2002; 74:725728; and Deng et al., Nano Lett 2003; 3:1545-8). A reason for the variable degree of templating might be the fact that variable binding sites on the DNA might be present due to the possible inconsistencies in the roughness of the substrate prepared by the LbL method.

Pre-Templated DNA with MNP.

In an effort to develop a more efficient templating strategy a series of experiments were performed to pretemplate long DNA molecules with MNP in solution. The best templating of the DNA molecules in solution was obtained when a solution of unfiltered MNP:DNA=5:1 (v:v) was prepared and “aged”. The ratio used was obtained empirically by testing many different combinations. To verify that this procedure results in DNA molecules templated with MNP, a drop of the solution was placed on a PSS terminated surface, dried with nitrogen and TMAFM images collected. Typical results from this type of experiment reveal a “network” of DNA-templated MNP. Based on a number of control experiments it has been determined that this type of structure is mediated by the presence of certain amount of DNA in the solution with MNP. Under the conditions of the present experiment the DNA suspended in solution is negatively charged and the TMAH-stabilized particles are positively charged. Thus the resulting network of pre-templated DNA molecules with MNP is due the electrostatic interactions in solution. Similar network structures were obtained by others when Au nanoparticles were used (Harnack et al., Nano Lett 2002; 2:919-23. Individual DNA molecules are not distinguishable from the TMAFM images, however one can find junctions where strings of MNP wires intersect. Images of less densely packed part of the surface clearly displayed wires composed of MNP with variable thicknesses and certain degree of particle aggregation. Upon filtration of the solution through an appropriate filter one can remove wires onto which large aggregates have formed and collect a solution of pretemplated DNA with MNP with a thickness of 7.5±1.2 nm. Wires using DNA as a template with similar thickness have been used in a number of proof of concept experiments where prototype nano-devices have been fabricated (see Keren et al., Science 2003; 302:1380-2).

MNP Templating of Stretched and Aligned DNA Via DPN Patterns Composed of PAH.

Electrostatic interactions were the one significant reason why we were able to template the DNA molecules with positively charged MNP in both types of experiments described in this Example. In subsequent steps, electrostatic interactions were relied upon to place and fabricate 1D structures on SiOx surfaces. Initially, DPN patterns were generated using PAH as an ink on SiOx substrates. Subsequently, molecular combing was performed on these lithographically defined surfaces. The presence of the features generated by DPN directs the placement of the DNA molecules on the surface. The direction at which they are extended is governed by the direction of the drop movement. Thus, in the majority of the experiments conducted, the DNA was stretched in between the lines of PAH upon combing due to they way the solution drop was moved across the surface. After examination by TMAFM to confirm the success of the combing procedure, the sample was exposed to a solution of MNP. The solution was obtained from the filtered MNP, synthesized as described above, and diluted with TMAH (MNP:TMAH=1:10=v:v). A deposition time of 5 min was used, the samples were rinsed with water to remove excess salt and surfactant, and dried under a stream of nitrogen.

TMAFM height and phase images, show a stretched and templated DNA strand in between the lines of the DPN template. The height image clearly shows the elongated DNA molecule that has successfully served as a template for the assembly of MNP. The line profiles from regions without and with MNP on the DNA molecule display an appreciable difference. For one particular case, the measured height of the “bare” DNA molecule was 0.78 nm and the height of the DNA templated with MNP was 2.1 nm. The scan parameters were optimized in order to collect high quality topography images, and in a number of cases the phase images acquired simultaneously were lacking contrast. However, in all cases the phase image was successfully used to identify the position specific templating of the DNA with MNPs. The experiments with DNP templates containing PAH resulted in more DNA molecules that were partially templated with MNP, compared to DNA molecules that were randomly combed on a PAH-terminated surface and subsequently templated with MNP. A possible reason for this result is the variable degree of PAH roughness on the two surfaces. In accordance with one aspect of the present invention a DPN protocol has been developed, based on electrostatic interactions, that allows for a consistent delivery of the PAH polyelectrolyte to the SiOx surface.

Alignment of Pre-Templated DNA with MNP Via DPN Patterns Composed of PSS.

To generate templates composed of PSS features SiOx substrates were patterned via DPN using PAH as an ink. Subsequently, a layer of PSS was adsorbed onto the PAH features using a LbL protocol. The line widths of the initial PAH features ranged from 65 to 250 nm and were maintained upon the adsorption of PSS. An increase in the thickness of the structures of 0.73±0.07 nm was measured from three randomly drawn profiles across a given line pattern. Initially, these templates were used to assemble pretemplated DNA with MNP. A surface containing DPN templates composed of squares patterns terminated with a PSS layer was used for the combing of the pre-templated DNA with MNP. The TMAFM images collected after the combing procedure indicated a successful placement of the pre-templated DNA with MNP. The measured height of the pre-templated DNA with MNP was 3.7±0.76 nm, based on three randomly drawn line profiles. No non-specific adsorption of the pre-templated DNA with MNP to the non-patterned SiOx was observed. Due to an initial lack of reproducible control over the elongation direction, the pre-templating conditions were adjusted so that the DNA molecule is still coated with MNP but flexible enough to allow for experiments where the stretching mechanism is similar to that of stretching “bare” DNA molecules. More particularly, the use of filtered particles allowed for the templating of DNA with MNP and its successful use in stretching experiments.

In the improved pre-templating procedure the MNP solution used was composed of particles with average size of 1.9±0.1 nm. The MNP stabilized by TMAH were diluted 1000 times and subsequently mixed with the DNA solution using the procedure described above. FIGS. 5a and 5b show the height and the phase image, respectively, of a DPN template composed of lines terminated on PSS prior to the combing procedure. Typical templates utilized in this part of the study had lines with widths of 65-200 nm separated by 1 mm distances. Using a pre-defined micron sized marker on the surface the pre-templated DNA with MNP was combed in a direction parallel to the lines. TMAFM images reveal that there are DNA-templated MNP attached onto areas where the PSS lines were placed, FIG. 5c. The pre-templated DNA with MNP in these areas formed strings of nanoparticles that were separated by about 1 mm distances. The strings of MNP lines that can be identified in FIG. 5c image have widths of 19.5±0.90 nm and heights of 3.2±0.32 nm. The width, without taking into effect any convolution effects induced by the tip, is considerably smaller than the original width of the DPN generated structures.

Unlike the results obtained by Nakao and co-workers where Au nanoparticles were assembled onto a template of DNA molecules (Nano Lett 2003; 3:1391-4), MNP were spaced non-homogenously along the length of the DNA. It is anticipated that a more homogenous assembly can be achieved if particles with better size distribution are used. Since the DNA was capable of being stretched in a desired direction based on an established molecular combing protocol, this suggests that the stretching mechanism of the pre-templated DNA with MNP is likely similar to that of stretching bare DNA molecules. In this mechanism, it is likely that one end of the templated DNA molecule, binds via one or multiple MNP to the negatively charged surface features composed of PSS and is flexible enough to be stretched by the moving air-solution interface during the combing procedure.

Although we were unable to image the individual DNA molecules our other results support the notion that the observed strings of MNP are placed in a location due to the presence of the DPN template and their arrangement is mediated by the presence of the DNA molecules used as a template in solution, prior to the combing procedure. To support this idea one can examine the edge of the DPN template—that is the area where the DPN patterns end and one images areas of unmodified SiOx, FIG. 5d. On the right side of FIG. 5d one can identify the last two lines of the DNP template with strings of pre-templated DNA with MNP. On the left side of the same figure, in the area of unmodified and non-patterned SiOx, a high degree of non-specifically bound and randomly oriented MNP can be observed. In a separate study, control experiments were performed where the DPN templates with PSS features were exposed to different concentrations of MNP solutions without the presence of DNA. At low concentrations of MNP, very few particles remained attached to any part of the surface upon washing. However, when higher MNP concentrations were utilized, a higher density of nanoparticles onto the entire length and width of the PSS patterns was observed. The degree of non-specific binding to the un-blocked SiOx surface was significant.

As shown herein elongated DNA molecules on templates generated via DPN can be templated with Fe₃O₄ nanoparticles. Furthermore, an aqueous procedure can be used to pretemplate DNA molecules with MNP, and such pretemplated DNAc can be stretched in a site specific manner by utilizing a template generated via DPN and molecular combing.

### Example 3

Magnetic Wires with DNA Cores

The development of methods to position, manipulate and image single nanoparticles is important from a fundamental physical as well as applied standpoint. Magnetic nanoparticles (NPs) have a wide range of technological applications in various fields, from storage media to drug delivery. Lithographic methods have been implemented to directly position magnetic NPs onto substrates. In recent years the use of biological molecules as templates or scaffolds for self-assembly processes has emerged as another promising route. DNA molecules have been utilized in molecular lithography that has resulted in working devices. Researchers have reported studies where DNA templates were used to fabricate nanowires. Such methods were limited to a few kinds of NPs (e.g. gold) or have relied on metallization. The techniques' utility can be extended by assembling magnetic NPs onto DNA templates. To produce the magnetic wires, one can dissolve single stranded λ-phage DNA (or othere DNA sequence) in buffer in order to use the long DNA molecules as a negatively charged scaffold.

Subsequently, appropriately functionalized water soluble Fe₃O₄ NPs with a positively charged shell can be introduced and allowed to self-assemble onto the DNA. This process results in the fabrication of long magnetic wires. Herein, we report a Magnetic Force Microscopy (MFM) investigation of the templated DNA. This work demonstrates that single stranded DNA molecules can act as excellent molecular scaffolds for the production of nanowires with magnetic properties.

The Fe₃O₄ NPs were prepared following a one-pot reaction. The synthesis is performed by refluxing 2.0 mmol of Fe(acac)₃ in 20 mL of 2-pyrrolidinone at 255° C. under a nitrogen atmosphere for 10 min. The solution was cooled to room temperature and the addition of methanol caused the precipitation of water soluble Fe₃O₄ particles. The methodology was verified by TEM, XPS, and FT-IR, and confirmed the expected size, composition, and the coordination of the carbonyl oxygen of the 2-pyrrolidinone to the surface Fe atoms of the NPs. The average size of the NPs, as determined by Tapping Mode Atomic Force Microscope (TMAFM), was 4.1±0.9 nm and represents a slight enlargement due to tip convolution effects compared with TEM values. The Fe₃O₄ particles were ferromagnetic according to SQUID measurements, and formed a stable solution for up to 2 months. The non-methylated λ-phage DNA from E. coli host strain GM 119 was dissolved in 10 mM Tris(hydroxymethly)aminomethane buffer. The wires were formed by incubating water solutions of NPs with DNA solutions in buffer for 30 minutes with mild shaking. The concentration ratios were 5 ng/μL:5 ng/μL (DNA:Fe₃O₄ NPs). The electrostatic attraction between the DNA and the particles in solution caused the particles to coat the DNA uniformly.

To evaluate the process, the coated DNA was stretched onto a silicon oxide surface via molecular combing. The silicon oxide was cleaned in solution of H₂SO₄:H₂O₂ (3:1; v:v) prior to the combing procedure. The solution was delivered to the surface of the substrate using a microliter syringe and an XYZ translation stage was used to slowly (30-50 μm/s) move the sample while keeping the solution suspended between the needle of the syringe and the substrate surface. The samples were characterized with MultiMode™ SPM from Digital Instruments. TMAFM images revealed the stretched and uniformly coated DNA molecules. Wires as long as 35 μm were observed. Results where up to ˜70% of the DNA present on the surface was coated with Fe₃O₄ NPs were consistently reported. The heights of the bare DNA and NP coated DNA were 0.8 nm and 2.5 nm, respectively. The average size deviation of the NPs assembled onto DNA-template, from the average size of the NPs in solution was due to the higher mobility of the smaller particles. Different DNA:Fe₃O₄ NPs ratios (i.e. 10:1, 5:1, and 1:5) resulted in partially coated DNA.

MFM measurements were done using the lift mode of the MultiMode™ SPM, where the tip makes two passes over the sample surface. In the first pass the tip lightly taps across the surface to generate a height image. In the second pass the tip is lifted at a certain distance away from the sample surface and traces the same line as in the height image. During this second pass the tip experiences a shift in its resonance frequency due to the force gradient between the tip and the sample. 15 In MFM the tip is magnetized thus the frequency shift is caused by a magnetic force. We used a silicon tip coated with a thin film of CoCr (Model MESP, Veeco, Inc). The tip was magnetized in a direction perpendicular to the sample surface. All the MFM imaging conditions were maintained constant for each set of experiments to minimize the effect of the magnetic NPs being magnetized with the stray field of the tip. The resonance frequency shift is the most direct indication of the change in force gradient between the tip and the sample. The cantilever resonance frequency shift was recorded using a frequency modulation where the cantilever's phase lag relative to the drive was kept at 90 degrees via a feedback.

Different lift heights were implemented, ranging from 5 nm to 50 nm. The frequency shift-versus-lift height graph for DNA coated with NPs with sizes from 1.5-8 nm showed that at lift heights of above 20 nm the frequency shift is very small and expected based on the weak magnetic properties of the NPs. The individual magnetic NPs are observed in both the topography and MFM images at lift distances below 10 nm. At that lift height the attractive van der Waals force becomes significant. Therefore, to rule out a possibility that we are not collecting a non-contact topographic images we used clean silicon tips in the MFM experiments and were not able to obtain any stable images.

Some of the topographic features are not pronounced as much as NPs in the MFM images. Therefore, it is believed that the major force responsible for the MFM contrast is indeed the magnetic force rather than van der Waals force. The contrast of the particles was always bright (dark) (without alternating high and low contrast) and was indicative of single domain particles with out of-plane magnetization under attractive (repulsive) interaction. Similar results were obtained with 75 nm×120 nm magnetic dots. The high coercivity of the tip and the size of the magnetic NPs with lower coercivity had strong effect on the contrast of the MFM images. The contrast of the MFM images was inverted at distances of 25 nm or higher when the magnetization direction of the tip was reversed. The magnetization of the tip is reversed by flipping the position of the magnetizing magnet with respect to the tip. When this is done the polarity of the magnet is changed, and the individual magnetic NPs are resolved. The separation between the particles ranged between 25-45 nm. The initial magnetization of the tip showed repulsive force between the tip and the sample. This force was replaced by an attractive one when the tip magnetization was reversed. This implies that the stray field from the tip was not affecting the magnetic states of the NPs in the previous experiments. We observed that this is no longer true at lower distances. At lower distances the stray field of the tip is affecting the NPs magnetic states.

In the present experiments, all the NPs, assembled on DNA and nonspecifically adsorbed on the surface, exhibited the bright contrast in MFM images showing an attractive interaction between the tip and the NPs. We postulate that the stronger field from the tip can change the magnetization states of the whole NPs' domains when the magnetization direction of the tip is reversed. These results were observed repeatedly back and forth, and are important since an ability to reverse the magnetization of individual bits is desirable in future bio-inspired magnetostatic devices.

In conclusion, the ability to assemble closely spaced Fe₃O₄ magnetic NPs on DNA templates has been demonstrated. The MFM studies showed that the DNA-templated Fe₃O₄ NPs form a densely packed magnetic wire. The assembled Fe₃O₄ NPs with sizes below 10 nm behave as single domain particles with an out-of-plane magnetization. Preliminary results show that the magnetization states of the assembled NPs on the DNA scaffold can be inverted and thus show promise to be placed in biologically inspired magnetostatic devices.

### Example 4

Enzymatic Cleavage of Templated DNA Molecules

It has recently been reported that DNA can be stretched onto solid surfaces via the surface tension of a receding meniscus of a DNA solution and the air/solution interface, and that such stretched DNA molecules maintained their biological activity and accessibility. More particularly, such stretched DNA can be enzymatically digested using a mix of restriction endonucleases (see Wang, Y. Huff, E. Schwartz, D. Proc. Natl. Acad Sci, 1995, 92, 165-169). The present experiments demonstrate the ability of DNA to electrostatically assemble magnetic nanoparticles while retaining its biochemical recognition properties, including the ability to be cleaved by restriction endonucleases such as BamH1.

To prepare DNA samples a 5 ng/mL solution of single-stranded non-methylated λ-phage DNA (E. coli host strain GM 119 from Sigma-Aldrich) in 10 mM Tris(hydroxymethyl)amino-methane (Tris) buffer was incubated with 5 ng of Fe₂O₃ magnetic nanoparticles. The mixture was mildly agitated on a vortex mixer for one hour at room temperature (22° C.). Prior to preparing the mixture, positively charged water soluble Fe₂O₃ magnetic nanoparticles were synthesized using a method described in Example 2. The average size of the particles as determined by Tapping Mode AFM (TMAFM) was 4.1±0.9 nm. The particles were also characterized using XPS, FTIR, TEM and XRD. The weak electrostatic interactions between the positively charged Fe₂O₃ nanoparticles and the negatively charged DNA caused the particles to align along the DNA. The resulting templated DNA was stretched onto freshly cleaned silicon oxide (WaferNet, CA) using molecular combing and subsequently rinsed with ultrapure water to remove any residual salts from the buffer before analysis by TMAFM.

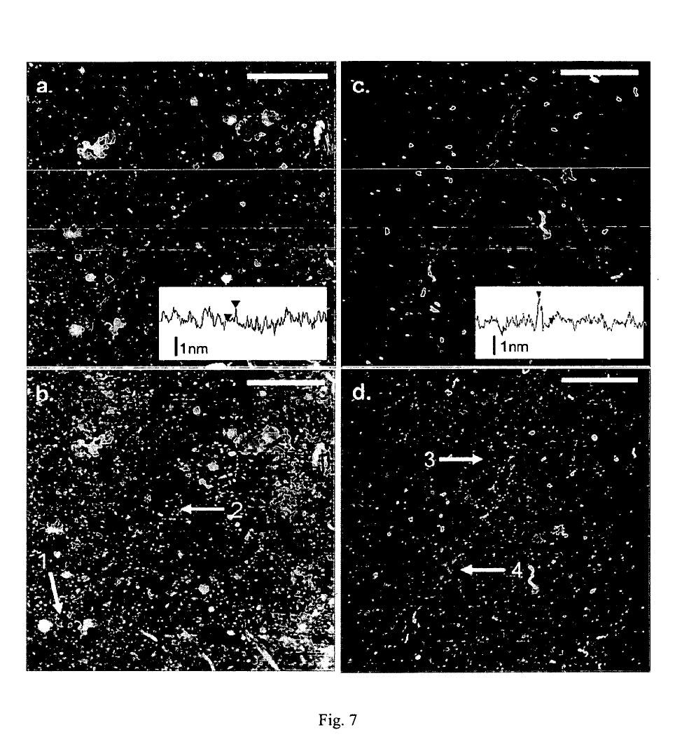

A typical experiment would yield SiO_{X} surfaces with varying concentrations of stretched DNA that was coated with nanoparitcles, (see FIGS. 6a and 6b). A line profile across a bare DNA yields a height of 0.76±0.10 nm, while a line scan across a DNA coated with magnetic nanoparticles yields a height of 1.6±0.2 nm. The difference in height between the particle coated DNA and the average size of nanoparticles is believed to be caused by the higher mobility and electrostatic interaction of the smaller particles to the DNA. Furthermore, the experiments showed that if one uses larger particles, the templated DNA cannot be easily stretched on surfaces.

The present experiments described herein demonstrate that stretched, surface immobilized DNA that has been templated with nanoparticles retains its recognition properties. Prior to performing experiments on the templated DNA, as an initial step it was verified that DNA immobilized on a modified SiO_{X} surface can be clipped with BamH1, FIGS. 7a and 7b. Arrows 1 & 2 show representative spots where the DNA was clipped by the enzyme in order to create gaps in the DNA strand. In a similar fashion, surfaces containing templated and stretched DNA were then treated with 35 μL of BamH1 enzyme (Sigma-Aldrich) at a concentration of 10 units for periods of 10 mins or less. After the enzyme treatment, the samples were again briefly rinsed with ultrapure water. FIGS. 7c and 7d show the stretched Fe₂O₃ templated DNA before and after clipping, respectively. It can be seen that the templated DNA strands, FIG. 7c, are digested in several spots, FIG. 7d. Representative areas where the enzyme has digested a region are indicated by arrows 3 & 4.

An important finding from these experiments is that portions of the templated DNA can be detached from the surface after the clipping. This suggests that the electrostatic interactions between the positively charged nanoparticles used to template the DNA and the negatively charged oxide surface are not strong enough to hold smaller fragments of the templated DNA. One reason for this result might be the fact that the strand is not coated uniformly and as it is stretched there are not an equal number of binding points to the surface along the length of the entire templated DNA molecule. Biological molecules are being actively explored as possible templates and scaffolds for nanotechnology applications and so far the progress has been promising. As shown herein, restriction enzymes represent an additional tool for DNA templated nanotechnology. More particularly, the BamH1 restriction enzyme can be used to fragment DNA after it has been templated with nanoparticles and has been immobilized and stretched on a surface. This technique may be successfully incorporated in higher order device structure nanofabrication and in the integration of bionanotechnology with microfabrication techniques. Future work will include controlling the gap size created by the enzyme clipping and in situ AFM analysis in fluid with BamH1 and other restriction enzymes.

1. A method of aligning nucleic acid molecules in a predetermined configuration on a solid surface, said method comprising the steps of
localizing the nucleic acid molecules on a nanotemplate formed on a solid surface; and
controlling the direction the nucleic acid molecules are elongated to provide elongated nucleic acid molecules in a predetermined configuration.

2. The method of claim 1 wherein the nucleic acid sequence is DNA.

3. The method of claim 2 wherein the nanotemplate comprises a polycation, and said nanotemplate is formed using dip-pen nanolithography.

4. The method of claim 3 wherein said DNA molecules are coated with a plurality of nanoparticles, said method further comprising the step of coating said nanotemplate with a polyanion prior to localizing the DNA molecules on the nanotemplate.

5. The method of claim 4 wherein the nanoparticles comprise metallic elements.

6. The method of claim 5 wherein the nanoparticles comprise magnetic material.

7. The method of claim 5 wherein the metallic elements are selected from the group consisting of Au, Ag, Cu, and Pt.

8. The method of claim 4 wherein the DNA molecules comprise a restriction endonuclease site, and the DNA is cleaved by said endonuclease after the DNA molecule is elongated on the template.

9. A kit comprising a DNA molecule that is coated with nanoparticles, said nucleic acid further comprising a restriction endonuclease recognition site.

10. The kit of claim 9 wherein the DNA molecule has a plurality of restriction endonuclease recognition sites positioned at predetermined distances from one another.

11. The kit of claim 9 wherein the nanoparticles comprise metallic elements.

12. The kit of claim 11 wherein the nanoparticles comprise magnetic nanoparticles.

13. The kit of claim 11 wherein the metallic elements are selected from the group consisting of Au, Ag, Cu, and Pt.

14. The kit of claim 11 further comprising a solid support and a nanotemplate bound to the solid support.

15. A composition comprising DNA molecules arranged on a nanotemplate, said naontemplate comprising a polyelectrolyte polymer bound to the semiconductor solid surface, wherein the DNA molecules have been elongated and positioned on the nanotemplate in a predetermined arrangement.

16. The composition of claim 15 wherein the DNA molecules have been coated with metallic nanoparticles.

17. The composition of claim 15 wherein the semiconductor solid surface comprises a group III-V or a group II-VI semi-conductor type material terminated with an oxide layer.

18. The composition of claim 17 wherein the nanotemplate comprises a polycation, and a layer of a polyanion coating the polycation.

19. A kit for aligning nucleic acid sequences on a solid surface, said kit comprising
a nucleic acid sequence comprising a plurality of nanoparticles bound to the nucleic acid sequence; and
a solution of a polycation, wherein the polycation is suitable for use in dip-pen nanolithography.

20. The kit of claim 19 wherein the nucleic acid sequence comprises an endonuclease restriction site located at a predetermined distance from a first end of the nucleic acid sequence.

21. The kit of claim 19 further comprising a solution of a polyanion.
